(19) United States
(12) Patent Application Publication
(10) Pub. No.: US 2008/0214862 A1
(43) Pub. Date: Sep. 4, 2008

(54) Processes for preparing armodafinil intermediate

(76) Inventors: Ben-Zion Dolitzky, Petach Tiqva (IL); Kobi Chen, Kfar-Saba (IL)

Correspondence Address: KENYON & KENYON LLP, ONE BROADWAY, NEW YORK, NY, 10004, US

(21) Appl. No.: 11/999,597

(22) Filed: Dec. 5, 2007

Related U.S. Application Data
(60) Provisional application No. 60/873,555, filed on Dec. 6, 2006.
(60) Provisional application No. 60/874,879, filed on Dec. 13, 2006.
(60) Provisional application No. 60/959,313, filed on Jul. 11, 2007.
(60) Provisional application No. 60/970,296, filed on Sep. 6, 2007.

(51) Int. Cl. C07C 317/10

(52) U.S. Cl. 562/429; 564/162

(57) ABSTRACT

The present invention encompasses processes for preparing intermediates of armodafinil, and the conversion of the intermediates to armodafinil.

### CROSS-REFERENCE TO RELATED APPLICATIONS

This application claims the benefit of U.S. provisional application Ser. Nos. 60/873,555, filed Dec. 6, 2006, 60/874,879, filed Dec. 13, 2006, 60/959,313, filed Jul. 11, 2007 and 60/970,296, filed Sep. 6, 2007, hereby incorporated by reference.

### FIELD OF THE INVENTION

The present invention is directed to racemization processes of S-modafinic acid or R-modafinic acid for the preparation of racemic modafinic acid (“BHSO”), an intermediate in the preparation of armodafinil, and to racemization processes of S-modafinil or R-modafinil.

### BACKGROUND OF THE INVENTION

Modafinil is currently marketed by Cephalon, Inc. under the trade name Provigil® as a racemic mixture of its R and S enantiomers. Provigil® is indicated for the treatment of excessive sleepiness associated with narcolepsy shift work sleep disorder (SWSD) and obstructive sleep apnea/hypopnea syndrome (OSA/HS).

Studies have shown that while both enantiomers of modafinil are pharmacologically active, the S enantiomer is eliminated from the body three times faster than the R enantiomer. T. Prisinzano et al., Tetrahedron: Asymmetry, vol. 5 (2004) 1053-1058. It is, therefore, preferable to develop pharmaceutical compositions of the R enantiomer of modafinil, as opposed to its racemic mixture.

The R enantiomer of modafinil is known as armodafinil and has the chemical name 2-[(R)-(diphenylmethyl)sulfinyl]acetamide. The molecular weight of armodafinil is 273.34 and it has the following chemical structure:

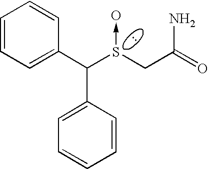

Armodafinil is known as Nuvigil™. Armodafinil was well tolerated and the safety profile was comparable with modafinil.

Armodafinil was first disclosed in the U.S. Pat. No. 4,927,855 (“'855 patent”) and its EP parallel patent No. 0233106, assigned to Lafon Laboratories.

The preparation of armodafinil is also disclosed in the '855 patent and it is summarized in the following scheme:

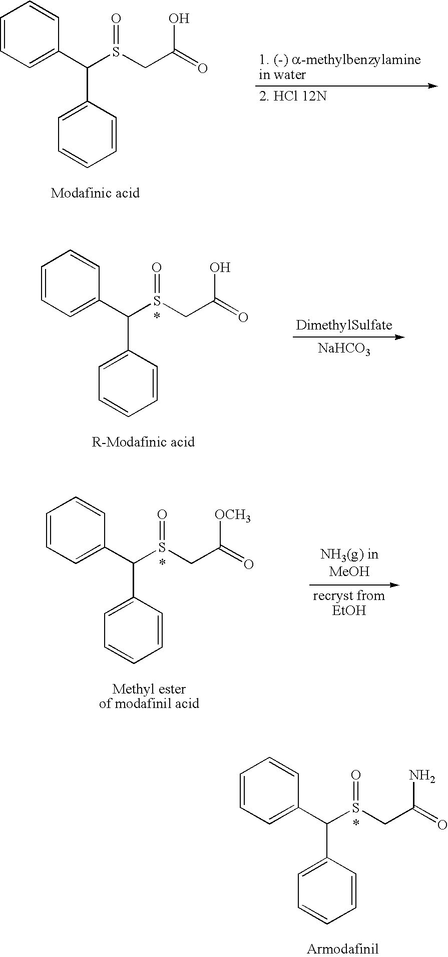

See '855 patent, col. 2, ll. 16-53.

In order to decrease the raw material cost, there is a need in the art to develop a process for the racemization of the undesired (S)-enantiomer of modafinic acid.

### SUMMARY OF THE INVENTION

In one embodiment, the invention encompasses a process for preparing racemic modafinic acid (“BHSO”) comprising combining S-BHSO or R-BHSO, an organic solvent and an acid promoter.

In another embodiment, the invention encompasses a process for preparing armodafinil by obtaining the racemic modafinic acid as described above, and converting R-modafinic acid to armodafinil.

In yet another embodiment, the invention encompasses a process for preparing racemic modafinil comprising combining armodafinil or S-modafinil and at least one organic solvent having a boiling point of above 60° C.; heating to a temperature of above 60° C.; and cooling.

In another embodiment, the invention encompasses a process for preparing armodafinil by obtaining the racemic modafinic acid as described above, and converting R-modafinic acid to armodafinil.

In one embodiment, the invention encompasses a process for preparing racemic BHSO comprising combining S-BHSO or R-BHSO and at least one organic solvent having a boiling point of above 60° C.; and heating to a temperature of above 60° C.; and cooling.

In another embodiment, the invention encompasses a process for preparing armodafinil by obtaining the racemic modafinic acid as described above, and converting R-modafinic acid to armodafinil.

### DETAILED DESCRIPTION OF THE INVENTION

The present invention encompasses processes for preparing intermediates of armodafinil, and the conversion of the intermediates to armodafinil.

As used herein, unless otherwise defined, the term “racemic modafinic acid” refers to 2-(benzhydryl-sulfinyl)acetic acid (“BHSO”) or a mixture of R/S-BHSO, wherein the mixture has both R- and S-BHSO in a ratio of 10:90 to 90:10 R-BHSO to S-BHSO, respectively.

As used herein, unless otherwise defined, the term “S-modafinic acid” refers to 2-(Benzhydryl S-Sulfinyl)acetic acid (“S-BHSO”).

As used herein, unless otherwise defined, the term “R-modafinic acid” refers to 2-(Benzhydryl R-Sulfinyl)acetic acid (“R-BHSO”).

As used herein, unless otherwise defined, the term “room temperature” refers to a temperature of about 15° C. to about 30° C., preferably about 18° C. to about 25° C.

The present invention encompasses a process for preparing racemic modafinic acid (“BHSO”) comprising combining S-BHSO or R-BHSO, at least one organic solvent, and an acid promoter.

Typically, the organic solvent is selected from the group consisting of C₁-C₄ alcohol, C₂-C₈ ester, C₄-C₈ ether, acetonitrile, C₃-C₈ ketone, C₁-C₈ chlorinated alkyl, and C₆-C₁₀ chlorinated aryl. Preferably, the C₁-C₄ alcohol is MeOH, EtOH, or isopropyl alcohol (IPA). Preferably, the C₂-C₈ ester is ethyl acetate, butyl acetate or isobutyl acetate. Preferably, the C₄-C₈ ether is tetrahydrofuran (“THF”), methyltetrahydrofuran (“MeTHF”) or cyclopentyl methyl ether. Preferably, the C₃-C₈ ketone is acetone or methyl isobutyl ketone (“MIBK”). Preferably, the C₁-C₈ chlorinated alkyl is dichloromethane. Preferably, the C₆-C₁₀ chlorinated aryl is chlorobenzene. Typically the organic solvent is present in an amount of about 50% to 95% by weight of the reaction mixture, and preferably in about 60% to 90% by weight of the reaction mixture.

The acid promoter may be at least one acyl halide or perchloric acid. Preferably, the acyl halide is C₁-C₈ acyl chloride. Typically, the C₁-C₈ acyl chloride is selected from the group consisting of: pivaloyl chloride, C₁-C₈ alkyl chloroformate, and C₁-C₈ chloroalkyl chloroformate. Preferably, the C₁-C₈ alkyl chloroformate is ethyl chloroformate. Typically, the acyl chloride is present in an amount of about 2 to 8 molar equivalents to the S or R-BHSO. Preferably, the acyl chloride is present in an amount of about 3 to 5 molar equivalents and more preferably in an amount of about 3 molar equivalents to the S or R-BHSO. Typically, the perchloric acid is present in an amount of about 10-30 molar equivalents to the S or R-BHSO. Preferably, the perchloric acid is present in an amount of 15 to 25 molar equivalents and more preferably, it is present in an amount of about 20-22 molar equivalents to the S or R-BHSO.

Typically, S-BHSO or R-BHSO, at least one organic solvent and at least one acid promoter are combined to obtain a reaction mixture and stirred for a time sufficient to obtain racemic BHSO. Preferably, the stirring is for about 3 hours. While stirring, the reaction mixture is maintained at a temperature of about −40° C. to about 50° C., and preferably, the temperature is at about room temperature.

Optionally, when an acyl halide is used, indium or indium salt is also added. In addition, when indium or indium salt is added, the organic solvent is not an alcohol. Indium and indium salts are typically used as catalysts in reactions. Preferably, the indium salt is selected from the group consisting of: InCl₃, InBr₃, and InI₃. When used indium or the indium salt is present in an amount of about 1 to 8 molar equivalents to the S-BHSO or R-BHSO, preferably it is present in an amount of about 1 to 4 molar equivalents, and more preferably the indium or indium salt is present in about 1.5 molar equivalents to the S-BHSO or R-BHSO.

Optionally, after the stirring step, at least one acid is added to the reaction mixture. Preferably, the acid is selected from the group consisting of: sulfuric acid, phosphoric acid and hydrochloric acid. After adding the acid, the reaction mixture is stirred for about 30 minutes to about 5 hours or until the hydrolysis step is complete. Preferably, the acid is present in an amount of about 5% to 10% by weight of the reaction mixture.

Optionally, water is added to the reaction mixture. Preferably, water is present in an amount of about 30% to 50% by weight of the reaction mixture.

Optionally, a mineral halide is added to the reaction mixture. In addition, when mineral halide is added, the organic solvent is not an alcohol. Typically, the mineral halide is a sodium salt or potassium salt. Preferably, the sodium salt is sodium bromide or sodium chloride. Preferably, the potassium salt is selected from the group consisting of: potassium fluoride, potassium bromide and potassium chloride. Preferably, the mineral halide is present in an amount of about 0.5% to 5% by weight of the reaction mixture. More preferably, both mineral halide and water are added to the reaction mixture when preparing racemic modafinic acid. Preferably, when indium or indium salt is added, water is not present in the reaction mixture.

After the addition of water and the mineral halide, the reaction mixture is further stirred at room temperature. Preferably, the reaction mixture is stirred for about 20 hours or until racemic modafinic acid is formed. Preferably, the reaction mixture is a clear solution or a suspension.

The racemic modafinic acid can be isolated from the reaction mixture, for example, by filtering or decanting. The solid may be washed and dried.

Preferably, the racemic modafinic acid produced by the process of the invention has about 10% to about 90% of the (R)-modafinic acid. More preferably, the racemic modafinic acid has about 40% to 60% of the (R)-modafinic acid.

The invention also encompasses a process for preparing armodafinil by obtaining racemic modafinic acid using the process described above, and converting it to armodafinil. A process for converting modafinic acid into armodafinil is exemplified in PCT Publication No. WO 07/103,221, hereby incorporated by reference. For example, armodafinil can be prepared by reacting R-modafinic acid with at least one acidic reagent and methanol or ethanol to obtain a solution of the C₁₋₂ ester R-modafinic and combining the solution of the ester with ammonia or ammonium hydroxide to obtain armodafinil. The resulting armodafinil may be isolated by any method known in the art, for example, precipitation followed by filtration, or solvent removal, such as evaporation, followed by trituration or recrystallization.

The invention further encompasses a process for preparing racemic modafinil comprising combining armodafinil or S-modafinil and at least one organic solvent having a boiling point of above 60° C.; and heating to a temperature of above 60° C., and cooling. Preferably, the organic solvent has a boiling point of above 100° C., and more preferably, of above about 190° C.

Typically, the organic solvent having a boiling point of above 60° C. is selected from the group consisting of: dimethylsulfoxide (DMSO), N,N-dimethylformamide (DMF), dimethylacetamide (DMA), xylene, toluene, C₄-C₁₀ alkyl acetate having boiling point of above 90° C., C₄-C₈ ketone, C₆-C₁₀ ether and a protic solvent having a boiling point of above 100° C. Preferably, the C₄-C₁₀ alkyl acetate has a boiling point of about 90° C. to about 140° C. Preferably, the C₄-C₈ ketone is methyl isobutyl ketone (MIBK). Preferably, the C₆-C₁₀ ether is cyclopentyl methyl ether (CPME). Preferably, the C₄-C₁₀ alkyl acetate having boiling point of above 90° C. is butyl acetate or isobutyl acetate. Preferably, the protic solvent having a boiling point of above 100° C. is C₄-C₈ alcohol. More preferably, the C₄-C₈ alcohol is dimethyl ethoxy ethanol or diethyl ethoxy ethanol. Preferably, the organic solvent is DMF or DMSO.

When armodafinil or S-modafinil and the organic solvent are combined, a reaction mixture is formed. Preferably, the reaction mixture is a suspension, a slurry, or a solution. Typically, the reaction mixture is heated to a temperature of not higher than 250° C. Preferably, the reaction mixture is heated to a temperature about 60° C. to 220° C. More preferably, the heating is to a temperature of above about 100° C., and most preferably to about 125° C. to about 180° C.

After the heating step, the reaction mixture is stirred for about 4 hours to about 48 hours or until the reaction is complete, and preferably, the reaction is stirred for about 17 hours.

The reaction mixture is preferably cooled to a temperature of less than about 50° C., and more preferably, to about 25° C.

The racemic modafinil may be isolated from the reaction mixture using any method known in the art, for example, by filtering or decanting.

The invention also encompasses a process for preparing armodafinil by obtaining racemic modafinic acid or racemic modafinil using the processes described above, and converting them to armodafinil.

The invention further encompasses a process for preparing racemic BHSO comprising combining S-BHSO or R-BHSO and at least one organic solvent having a boiling point of above 60° C.; heating to a temperature of 60° C.; and cooling. The process parameters are the same as those described above in the process for preparing racemic modafinil.

The reaction times described herein are reaction times suitable for laboratory-scale preparations. One of ordinary skill in the art understands that suitable reaction times will vary based upon the amounts of reagents present, and can adjust the reaction times accordingly.

Having described the invention with reference to certain preferred embodiments, other embodiments will become apparent to one skilled in the art from consideration of the specification. The invention is further defined by reference with the following examples describing in detail the analysis of the armodafinil and intermediates thereof and methods for preparing thereof. It will be apparent to those skilled in the art that many modifications, both to materials and methods, may be practiced without departing from the scope of the invention.

### EXAMPLES

### HPLC Conditions

The reaction mixture was analyzed by HPLC using Chiralpak AD-H 4.6*150 mm DAIC 19324 as the column and packing. The parameters were eluent: 90 hexane: 10 IPA: 0.1 trifluoroacetic acid (TFA), flow rate: 1.5 ml/minute, detector: 225 nm, sample volume: 20 ml, column temperature: 25° C., auto sampler temperature: 25° C., and diluent: ethanol. Mobile phase composition and flow rate may be varied in order to achieve the required system suitability.

### Example 1

### A Process for Obtaining Racemic Modafinic Acid from (R)-Modafinic Acid

A 50 ml flask was charged with 2-(Benzhydryl R-Sulfinyl)acetic acid (R-BHSO, 0.81 g), iso-propanol (18 ml), elemental indium (0.61 g, 1.5 eq) and pivaloyl chloride (1.27 g, 3 eq). The suspension was stirred at room temperature for 3 hours to obtain a clear solution. HCl (32%, 1 ml) was then added and further stirred for 30 minutes. The sample was analyzed by HPLC to get 1.5% by area of S-BHSO.

### Example 2

### A Process for Obtaining Racemic Modafinic Acid from (R)-Modafinic Acid

A 50 ml flask was charged with 2-(Benzhydryl R-Sulfinyl)acetic acid (R-BHSO, 0.81 g), methanol (18 ml), elemental indium (0.61 g, 1.5 eq) and pivaloyl chloride (1.27 g, 3 eq). The suspension was stirred at room temperature for 3 hours to obtain a clear solution. HCl (32%, 1 ml) was then added and further stirred for 30 minutes. The sample was analyzed in HPLC to get 0.85% by area of S-BHSO.

### Example 3

### A Process for Obtaining Racemic Modafinic Acid from (R)-Modafinic Acid

A 50 ml flask was charged with 2-(Benzhydryl R-Sulfinyl)acetic acid (R-BHSO, 0.81 g), tetrahydrofuran (18 ml), elemental indium (0.61 g, 1.5 eq) and pivaloyl chloride (1.27 g, 3 eq). The suspension was stirred at room temperature for 3 hours to obtain a clear solution. HCl (32%, 1 ml) was then added and further stirred for 30 minutes. The sample was analyzed by HPLC to get 13.07% by area of S-BHSO.

### Example 4

### A Process for Obtaining Racemic Modafinic Acid from (R)-Modafinic Acid

A 50 ml flask was charged with 2-(Benzhydryl R-Sulfinyl)acetic acid (R-BHSO, 0.81 g), ethyl acetate (18 ml), elemental indium (0.61 g, 1.5 eq) and pivaloyl chloride (1.27 g, 3 eq). The suspension was stirred at room temperature for 3 hours to obtain a clear solution. HCl (32%, 1 ml) was then added and further stirred for 30 minutes. The sample was analyzed by HPLC to get 24.93% by area of S-BHSO.

### Example 5

### A Process for Obtaining Racemic Modafinic Acid from (R)-Modafinic Acid

A 50 ml flask was charged with 2-(Benzhydryl R-Sulfinyl)acetic acid (R-BHSO, 0.81 g), acetone (18 ml), elemental indium (0.61 g, 1.5 eq) and pivaloyl chloride (1.27 g, 3 eq). The suspension was stirred at room temperature for 3 hours to obtain a clear solution. HCl (32%, 1 ml) was then added and further stirred for 30 minutes. The sample was analyzed by HPLC to get 48.79% by area of S-BHSO.

### Example 6

### A Process for Obtaining Racemic Modafinic Acid from (R)-Modafinic Acid

A 50 ml flask was charged with 2-(Benzhydryl R-Sulfinyl)acetic acid (R-BHSO, 0.5 g), sodium bromide (0.3 g), water (10 ml), tetrahydrofuran (2 ml) and perchloric acid (6 ml, 70%). The suspension was stirred at room temperature for 20 hours. The sample was analyzed by HPLC to get 15.7% by area of S-BHSO.

### Example 7

### A Process for Obtaining Racemic Modafinic Acid from (R)-Modafinic Acid

A 50 ml flask was charged with 2-(Benzhydryl R-Sulfinyl)acetic acid (R-BHSO, 0.5 g), sodium chloride (0.1 g), water (10 ml), tetrahydrofuran (2 ml) and perchloric acid (6 ml, 70%). The suspension was stirred at room temperature for 20 hours. The sample was analyzed by HPLC to get 2.4% by area of S-BHSO.

### Example 8

### A Process for Obtaining Racemic Modafinic Acid from (R)-Modafinic Acid

A 50 ml flask was charged with 2-(Benzhydryl R-Sulfinyl)acetic acid (R-BHSO, 0.5 g), sodium bromide (0.3 g), water (10 ml), ethyl acetate (2 ml) and perchloric acid (6 ml, 70%). The suspension was stirred at room temperature for 20 hours. The sample was analyzed by HPLC to get 3.8% by area of S-BHSO.

### Example 9

### A Process for Obtaining Racemic Modafinic Acid from (R)-Modafinic Acid

A 50 ml flask was charged with 2-(Benzhydryl R-Sulfinyl)acetic acid (R-BHSO, 0.5 g), sodium bromide (0.1 g), water (10 ml), acetonitrile (2 ml) and perchloric acid (6 ml, 70%). The suspension was stirred at room temperature for 20 hours. The sample was analyzed by HPLC to get 1.9% by area of S-BHSO.

### Example 10

### Racemization of Armodafinil in DMSO

A 20 ml vial equipped with a magnetic stirrer was charged with Armodafinil (0.5 g) and dimethylsulfoxide (5 ml). The suspension was heated to 100° C. and was stirred for 17 hours to obtain yellow slurry and then cooled to 25° C. The sample was analyzed by HPLC to be 86.2% of R and 13.8% of S.

### Example 11

### Racemization of Armodafinil in DMF

A 20 ml vial equipped with a magnetic stirrer was charged with Armodafinil (0.5 g) and N,N-dimethylformamide (5 ml). The suspension was heated to 100° C. and was stirred for 17 hours to obtain orange slurry and then cooled to 25° C. The sample was analyzed by HPLC to be 90.6% by area of R and 9.4% by area of S.

### Example 12

### Racemization of S-BHSO in DMSO

A 20 ml vial equipped with a magnetic stirrer was charged with S-BHSO (0.5 g) and dimethylsulfoxide (5 ml). The suspension was heated to 90° C. and was stirred for 17 hours to obtain a yellow solution and then cooled to 25° C. The sample was analyzed by HPLC to be 80.0% by area of S and 20.0% by area of R.

### Example 13

### Racemization of S-BHSO in DMF

A 20 ml vial equipped with a magnetic stirrer was charged with S-BHSO (0.5 g) and N,N-dimethylformamide (5 ml). The suspension was heated to 90° C. and was stirred for 17 hours to obtain a yellow solution and then cooled to 25° C. The sample was analyzed by HPLC to be 86.0% by area of S and 14.0% by area of R.

We claim:

1. A process for preparing racemic 2-(benzhydryl-sulfinyl)acetic acid comprising combining 2-(benzhydryl S-sulfinyl)acetic acid or 2-(benzhydryl R-sulfinyl)acetic acid, at least one organic solvent and at least one acid promoter.

2. The process of claim 1, wherein the organic solvent is selected from the group consisting of: C₁-C₄ alcohol, C₂-C₈ ester, C₄-C₈ ether, acetonitrile, C₃-C₈ ketone, C₁-C₈ chlorinated alkyl and C₆-C₁₀ chlorinated aryl.

3. The process of claim 2, wherein the organic solvent is selected from the group consisting of: MeOH, EtOH, isopropyl alcohol, ethyl acetate, butyl acetate, isobutyl acetate, tetrahydrofuran, methyltetrahydrofuran, cyclopentyl methyl ether, acetone, methyl isobutyl ketone, dichloromethane, and chlorobenzene.

4. The process of claim 1, wherein the acid promoter is perchloric acid.

5. The process of claim 1, wherein the acid promoter is an acyl halide.

6. The process of claim 5, wherein the acyl halide is C₁-C₈ acyl chloride.

7. The process of claim 1, wherein the 2-(benzhydryl S-sulfinyl)acetic acid or the 2-(benzhydryl R-sulfinyl)acetic acid, at least one organic solvent and at least one acid promoter are combined to obtain a reaction mixture and stirred for a time sufficient to obtain racemic 2-(benzhydryl-sulfinyl)acetic acid.

8. The process of claim 1, wherein the process further comprises adding indium or at least one indium salt.

9. The process of claim 1, further comprising adding an acid.

10. The process of claim 9, wherein the acid is sulfuric acid, phosphoric acid or hydrochloric acid.

11. The process of claim 1, further comprising adding water.

12. The process of claim 1, further comprising adding a mineral halide.

13. The process of claim 12, wherein the mineral halide is a sodium salt or potassium salt.

14. The process of claim 1, wherein the racemic 2-(benzhydryl-sulfinyl)acetic acid contains about 10% to about 90% of the 2-(benzhydryl R-sulfinyl)acetic acid.

15. A process for preparing 2-[(R)-(diphenylmethyl)sulfinyl]acetamide comprising:
a) combining 2-(benzhydryl S-sulfinyl)acetic acid or 2-(benzhydryl R-sulfinyl)acetic acid, at least one organic solvent and at least one acid promoter to obtain a mixture containing racemic 2-(benzhydryl-sulfinyl)acetic acid; and
b) converting the racemic 2-(benzhydryl-sulfinyl)acetic acid to 2-[(R)-(diphenylmethyl)sulfinyl]acetamide.

16. A process for preparing racemic 2-[(diphenylmethyl)sulfinyl]acetamide comprising:
combining 2-[(R)-(diphenylmethyl)sulfinyl]acetamide or 2-[(S)-(diphenylmethyl)sulfinyl]acetamide, at least one organic solvent having a boiling point of above 60° C.; heating to a temperature of above 60° C.; and cooling.

17. A process for preparing racemic 2-(benzhydryl-sulfinyl)acetic acid comprising: combining 2-(benzhydryl S-sulfinyl)acetic acid or the 2-(benzhydryl R-sulfinyl)acetic acid, at least one organic solvent having a boiling point of above 60° C.; heating to a temperature of above 60° C.; and cooling.

18. The process of claim 16 or 17, wherein the organic solvent is selected from the group consisting of: dimethylsulfoxide, N,N-dimethylformamide, dimethylacetamide, xylene, toluene, C₁-C₈ alkyl acetate having boiling point of above 90° C., C₄-C₈ ketone, C₆-C₁₀ ether and a protic solvent having a boiling point of above 100° C.

19. The process of claim 18, wherein the organic solvent is selected from the group consisting of: methyl isobutyl ketone, cyclopentyl methyl ether, butyl acetate, isobutyl acetate, dimethyl ethoxy ethanol and diethyl ethoxy ethanol.

20. The process of claim 16 or 17, wherein the heating temperature is above about 60° C. to about 220° C.

21. The process of claim 16 or 17, wherein the cooling is carried out at a temperature below about 50° C.

22. A process for preparing 2-[(R)-(diphenylmethyl)sulfinyl]acetamide comprising:
a) combining 2-[(R)-(diphenylmethyl)sulfinyl]acetamide or 2-[(S)-(diphenylmethyl)sulfinyl]acetamide, at least one organic solvent having a boiling point of above 60° C.; heating to a temperature of above 60° C.; cooling and
b) converting the racemic 2-(diphenylmethyl-sulfinyl)acetamide to 2-[(R)-(diphenylmethyl)sulfinyl]acetamide.

23. A process for preparing 2-[(R)-(diphenylmethyl)sulfinyl]acetamide comprising:
a) combining 2-(benzhydryl S-sulfinyl)acetic acid or 2-(benzhydryl R-sulfinyl)acetic acid, at least one organic solvent having a boiling point of above 60° C.; heating to a temperature of above 60° C.; cooling and
b) converting the racemic 2-(benzhydryl-sulfinyl)acetic acid to 2-[(R)-(diphenylmethyl)sulfinyl]acetamide.
